(19) United States
(12) Patent Application Publication
(10) Pub. No.: US 2025/0288782 A1
(43) Pub. Date: Sep. 18, 2025

(54) METHOD OF REDUCING LEFT ATRIAL PRESSURE

(71) Applicant: EDWARDS LIFESCIENCES CORPORATION, Irvine, CA (US)

(72) Inventors: TARANNUM ISHAQ GUTIERREZ, Ladera Ranch, CA (US); Linda Thai, Mission Viejo, CA (US); Zachary Charles VanEvery, Lake Forest, CA (US); Denis TAuz, Laguna Hills, CA (US); Cooper Ryan Rickerson, Huntington Beach, CA (US)

(73) Assignee: EDWARDS LIFESCIENCES CORPORATION, Irvine, CA (US)

(21) Appl. No.: 19/227,359

(22) Filed: Jun. 3, 2025

Related U.S. Application Data
(63) Continuation of application No. 18/601,755, filed on Mar. 11, 2024.
Application No. 18/601,755 is a continuation of application No. 17/818,557, filed on Aug. 9, 2022.
Application No. 17/818,557 is a continuation of application No. PCT/US2021/016142, filed on Feb. 2, 2021.
(60) Provisional application No. 62/975,024, filed on Feb. 11, 2020.

(51) Int. Cl. A61M 27/00

(52) U.S. Cl. CPC A61M 27/002; A61M 2205/04; A61M 2210/125

(57) ABSTRACT

A method of reducing left atrial pressure is provided for treating diastolic heart failure. A puncture catheter is advanced into a coronary sinus for creating an opening in a wall between the coronary sinus and a left atrium of a heart. A cauterizing tool may be used to create a controlled scarring pattern in the tissue along the opening. After the opening is created, a blood flow path is provided from the left atrium to the coronary sinus for reducing blood pressure in the left atrium and thereby alleviating symptoms of heart failure. If necessary, a shunt device may be implanted within the opening to ensure that the blood flow path remains open.

### RELATED APPLICATION

This application is a continuation of U.S. application Ser. No. 18/601,755, filed on Mar. 11, 2024, which is a continuation of U.S. application Ser. No. 17/818,557, filed on Aug. 9, 2022, which is a continuation of International Application No. PCT/US2021/016142, filed on Feb. 2, 2021, which claims the benefit of U.S. Provisional Application No. 62/975,024, filed on Feb. 11, 2020, the disclosures of which are hereby incorporated by reference.

### BACKGROUND

The present invention relates generally to cardiac shunts and systems and methods of delivery, and in particular, to a shunt to reduce left atrial pressure.

Heart failure is a common and potentially lethal condition affecting humans, with sub-optimal clinical outcomes often resulting in symptoms, morbidity and/or mortality, despite maximal medical treatment. In particular, “diastolic heart failure” refers to the clinical syndrome of heart failure occurring in the context of preserved left ventricular systolic function (ejection fraction) and in the absence of major valvular disease. This condition is characterized by a stiff left ventricle with decreased compliance and impaired relaxation, which leads to increased end-diastolic pressure. Approximately one third of patients with heart failure have diastolic heart failure and there are very few, if any, proven effective treatments.

Symptoms of diastolic heart failure are due, at least in a large part, to an elevation in pressure in the left atrium. Elevated Left Atrial Pressure (LAP) is present in several abnormal heart conditions, including Heart Failure (HF). In addition to diastolic heart failure, a number of other medical conditions, including systolic dysfunction of the left ventricle and valve disease, can lead to elevated pressures in the left atrium. Both Heart Failure with Preserved Ejection Fraction (HFpEF) and Heart Failure with Reduced Ejection Fraction (HFrEF) can exhibit elevated LAP. It has been hypothesized that both subgroups of HF might benefit from a reduction in LAP, which in turn reduces the systolic preload on the left ventricle, Left Ventricular End Diastolic Pressure (LVEDP). It could also relieve pressure on the pulmonary circulation, reducing the risk of pulmonary edema, improving respiration and improving patient comfort.

### SUMMARY

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

Some implementations of the present disclosure relate to a shunt comprising a central flow portion configured to fit at least partially within an opening in a tissue wall. The tissue wall is situated between a first anatomical chamber and a second anatomical chamber and the opening creates a blood flow path between the first anatomical chamber and the second anatomical chamber. The central flow portion is further configured to maintain the blood flow path from the first anatomical chamber to the second anatomical chamber. The shunt further comprises a barrier configured to alter growth of tissue around the shunt.

The shunt may further comprise one or more anchoring arms extending from the central flow portion. The one or more anchoring arms may be configured to anchor to the tissue wall.

In some embodiments, the barrier extends from at least one of the one or more anchoring arms. The barrier may comprise one or more spikes extending from at least one of the one or more anchoring arms.

In some embodiments, the one or more spikes have pointed ends. The barrier may comprise a first spike and a second spike. The first spike may be configured to be situated further from the opening than the second spike.

In some embodiments, the barrier extends from the central flow portion. The barrier may comprise a first portion configured to extend over a first side of the tissue wall. In some embodiments, the first portion is configured to extend at an approximately 45-degree angle over the first side of the tissue wall.

The barrier may comprise a second portion configured to extend over a second side of the tissue wall. In some embodiments, the first portion and the second portion form a single continuous device.

The opening may have an elliptical shape and the first portion may form at least a partial cone with a tapered elliptical shape in which the first portion extends over a full ellipse of tissue on the first side of the tissue wall. In some embodiments, the first portion does not extend over the opening.

A length of the first portion may be greater than a width and thickness of the first portion. In some embodiments, the first portion may have a shape of an at least partial elliptical ring with a hollow middle portion configured to be aligned with the opening. The shunt may further comprise one or more anchoring arms extending from the central flow portion. The one or more anchoring arms may be configured to anchor to the tissue wall. In some embodiments, the barrier extends from at least one of the one or more anchoring arms. The barrier may be configured to be situated between the one or more anchoring arms and the tissue wall. In some embodiments, the central flow portion is further configured to prevent in-growth of tissue within the opening. The central flow portion may be configured to expand in response to expansion of the tissue wall.

Some implementations of the present disclosure relate to a method comprising creating an opening in a tissue wall, treating an area of tissue around the opening to prevent in-growth of tissue at the opening, and placing a shunt at the opening.

The area of tissue may have an elliptical shape and completely surround the opening. In some embodiments, the shunt comprises a central flow portion configured to fit at least partially within an opening in a tissue wall.

The tissue wall may be situated between a first anatomical chamber and a second anatomical chamber and the opening may represent a blood flow path between the first anatomical chamber to the second anatomical chamber. The central flow portion may be further configured to maintain the blood flow path from the first anatomical chamber to the second anatomical chamber.

In some embodiments, the shunt further comprises a barrier configured to alter growth of tissue around the shunt. Treating the area of tissue may involve burning the area of tissue.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements. However, it should be understood that the use of similar reference numbers in connection with multiple drawings does not necessarily imply similarity between respective embodiments associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some embodiments or configurations.

FIG. 1 illustrates several access pathways for maneuvering guidewires and/or catheters in and around the heart to deploy expandable shunts in accordance with some embodiments.

FIG. 2 depicts a method for deploying expandable shunts in accordance with some embodiments.

FIG. 3A is a side view of an opening through a tissue wall for placement of a shunt in the opening in accordance with some embodiments.

FIG. 3B is a view from above (e.g., from the left atrium) of an opening through a tissue wall for placement of a shunt in the opening in accordance with some embodiments.

FIG. 4 illustrates a shunt having one or more barrier walls to prevent, contain, and/or inhibit tissue growth at and/or around the shunt and/or an opening in a tissue wall in accordance with some embodiments.

FIG. 5 illustrates a shunt having one or more barrier spikes to prevent, contain, and/or inhibit tissue growth in accordance with some embodiments.

FIG. 6 illustrates a method of preventing, inhibiting, and/or containing tissue growth involving treating an area of tissue around an opening in accordance with some embodiments.

FIG. 7 illustrates a shunt having an upper barrier to prevent, contain, and/or inhibit tissue growth in accordance with some embodiments.

FIGS. 8A illustrates a side-view of a shunt having a lower barrier to prevent, contain, and/or inhibit tissue growth in accordance with some embodiments.

FIGS. 8B illustrates a view from above (e.g., from the left atrium) of a shunt having a lower barrier to prevent, contain, and/or inhibit tissue growth in accordance with some embodiments.

FIG. 9 is a flow diagram of an example of a process for delivering and/or anchoring a shunt to a body of a person to in accordance with some embodiments.

### DETAILED DESCRIPTION

The headings provided herein are for convenience only and do not necessarily affect the scope or meaning of the claimed invention.

### Overview

In vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and are each mounted in an annulus comprising dense fibrous rings attached either directly or indirectly to the atrial and ventricular muscle fibers. Each annulus defines a flow orifice. The four valves ensure that blood does not flow in the wrong direction during the cardiac cycle; that is, to ensure that the blood does not back flow through the valve. Blood flows from the venous system and right atrium through the tricuspid valve to the right ventricle, then from the right ventricle through the pulmonary valve to the pulmonary artery and the lungs. Oxygenated blood then flows through the mitral valve from the left atrium to the left ventricle, and finally from the left ventricle through the aortic valve to the aorta/arterial system.

Heart failure is a common and potentially lethal condition affecting humans, with sub-optimal clinical outcomes often resulting in symptoms, morbidity and/or mortality, despite maximal medical treatment. In particular, “diastolic heart failure” refers to the clinical syndrome of heart failure occurring in the context of preserved left ventricular systolic function (ejection fraction) and in the absence of major valvular disease. This condition is characterized by a stiff left ventricle with decreased compliance and impaired relaxation, which leads to increased end-diastolic pressure. Approximately one third of patients with heart failure have diastolic heart failure and there are very few, if any, proven effective treatments.

Symptoms of diastolic heart failure are due, at least in a large part, to an elevation in pressure in the left atrium. Elevated Left Atrial Pressure (LAP) is present in several abnormal heart conditions, including Heart Failure (HF). In addition to diastolic heart failure, a number of other medical conditions, including systolic dysfunction of the left ventricle and valve disease, can lead to elevated pressures in the left atrium. Both Heart Failure with Preserved Ejection Fraction (HFpEF) and Heart Failure with Reduced Ejection Fraction (HFrEF) can exhibit elevated LAP. It has been hypothesized that both subgroups of HF might benefit from a reduction in LAP, which in turn reduces the systolic preload on the left ventricle, Left Ventricular End Diastolic Pressure (LVEDP). It could also relieve pressure on the pulmonary circulation, reducing the risk of pulmonary edema, improving respiration and improving patient comfort.

Pulmonary hypertension (PH) is defined as a rise in mean pressure in the main pulmonary artery. PH may arise from many different causes, but, in all patients, has been shown to increase mortality rate. A deadly form of PH arises in the very small branches of the pulmonary arteries and is known as Pulmonary Arterial Hypertension (PAH). In PAH, the cells inside the small arteries multiply due to injury or disease, decreasing the area inside of the artery and thickening the arterial wall. As a result, these small pulmonary arteries narrow and stiffen, causing blood flow to become restricted and upstream pressures to rise. This increase in pressure in the main pulmonary artery is the common connection between all forms of PH regardless of underlying cause. Despite previous attempts, there is a need for an improved way to reduce elevated pressure in the left atrium, as well as other susceptible heart chambers such as the pulmonary artery.

The present disclosure provides methods and devices that may allow for elevated LAP to be reduced by shunting blood from a first anatomical chamber (e.g., the left atrium) to a second anatomical chamber (e.g., the coronary sinus). While some embodiments herein may be described with respect to treating LAP and/or similar issues, the shunting devices and methods described may be used to treat other issues, including dialysis. Some embodiments involve a shunt defining an open pathway between the left atrium and the coronary sinus, although the method can be used to place a shunt between other cardiac chambers, such as between the pulmonary artery and right atrium. The term “shunt” is used herein according to its broad and ordinary meaning and may include any shunting means and/or means for shunting blood between and/or from one anatomical chamber and/or blood pathway to another anatomical chamber and/or blood pathway. The shunt may be configured to prevent initial collapse of the open pathway while also preventing in-growth of tissue at least at an inner surface of the open pathway. In some embodiments, the shunt may be expandable so as to be compressed, delivered via a low-profile sheath or tube, and expelled so as to resume its expanded state. Some methods may also include utilizing a deployment catheter that may first create a puncture in a tissue wall between the left atrium and the coronary sinus. A catheter as described herein can include any delivery means and/or means for delivering one or more implants within a body of a patient.

Moreover, in some embodiments, a shunt may be configured to expand post-delivery in response to expansion of the tissue wall. For example, some patients, and particularly HF patients, may experience amyloidosis, which is a protein disorder in which amyloid deposits in the heart can make the heart walls stiffen and/or increase in thickness. Shunt implants having a maximum tissue wall thickness specification may not be configured to accommodate some levels of tissue growth/expansion. For example, some shunt implants may have wall thickness specifications of approximately 4 mm. However, many amyloidosis patients can have tissue wall thickness that may continue to increase beyond 4 mm, therefore causing patency issues with shunt implants post-implantation. Accordingly, it may be advantageous for shunt implants to include features configured to present a physical barrier to tissue growth and/or for shunt implants to be delivered in association with devices and/or methods for preventing and/or inhibiting tissue growth. For example, a barrier may include a wall, spike, ring, or other device which may extend from and/or attach to a shunt to prevent, inhibit, and/or contain tissue growth at and/or around the shunt and/or the opening in the tissue wall. In some embodiments, shunt implants may also be at least partially expandable and/or configured to “grow” in response to tissue growth.

Shunt implants described herein may therefore include a central flow portion and/or anchoring arms that may be configured to attach to various mechanical elements configured to prevent and/or inhibit tissue growth. An anchoring arm may include any anchoring means and/or means for anchoring a shunt implant and/or portion of a shunt implant. In some embodiments, shunt implants may be delivered using methods configured to prevent and/or inhibit tissue growth around and/or near the shunt implants. Shunt implants may incorporate various mechanical systems to prevent and/or inhibit tissue growth. Details of these methods, implants and deployment systems will be described below.

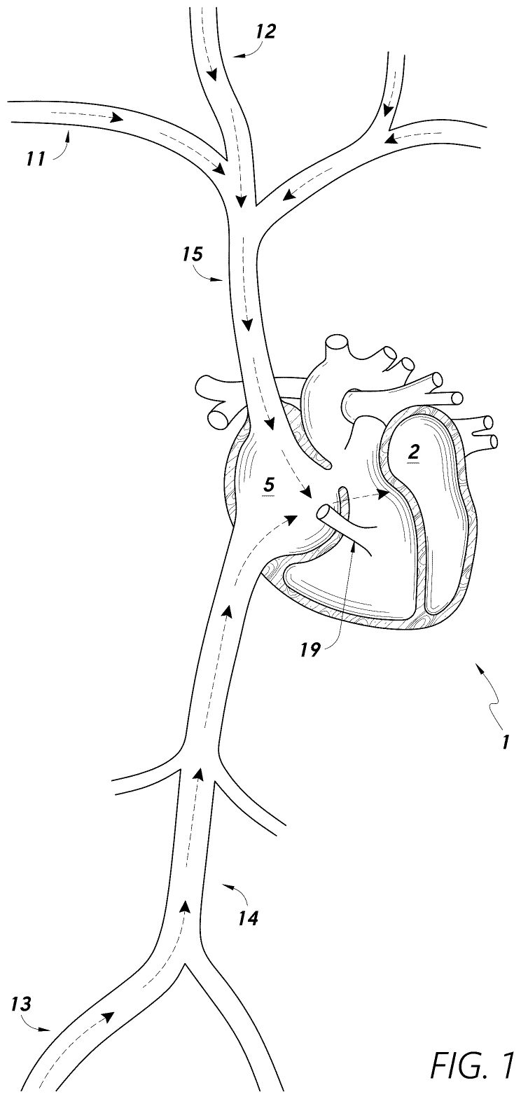

FIG. 1 illustrates several access pathways for maneuvering guidewires and catheters in and around the heart 1 to deploy expandable shunts of the present application. For instance, access may be from above via either the subclavian vein 11 or jugular vein 12 into the superior vena cava (SVC) 15, right atrium (RA) 5 and from there into the coronary sinus (CS) 19. Alternatively, the access path may start in the femoral vein 13 and through the inferior vena cava (IVC) 14 into the heart 1. Other access routes may also be used, and each typically utilizes a percutaneous incision through which the guidewire and catheter are inserted into the vasculature, normally through a sealed introducer, and from there the physician controls the distal ends of the devices from outside the body.

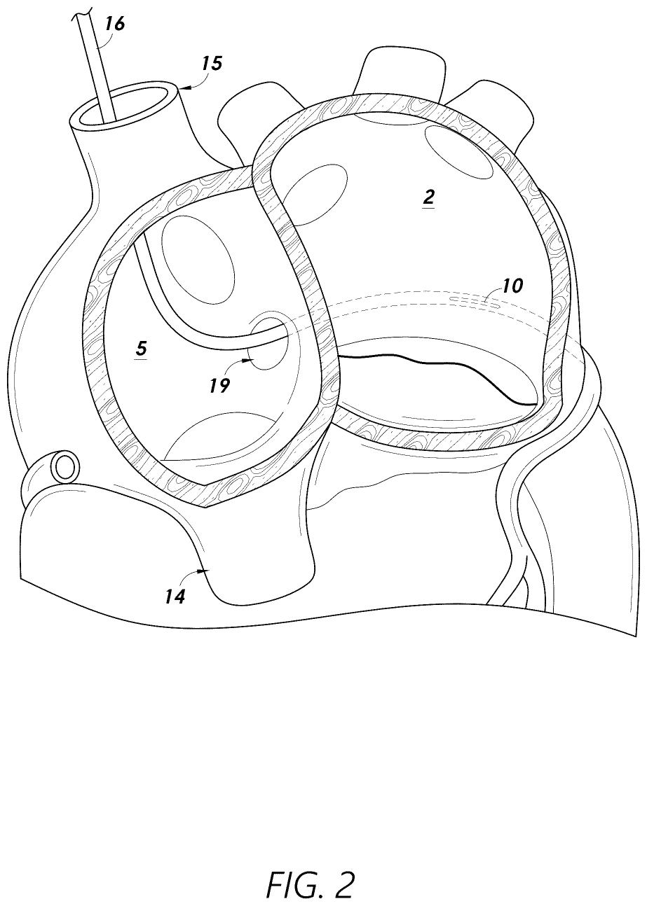

FIG. 2 depicts a method for deploying various implants 10 including expandable shunts described herein, wherein a catheter 16 is introduced through the subclavian or jugular vein, through the SVC 15 and into the coronary sinus 19. Once a guidewire provides a path, an introducer sheath may be routed along the guidewire and into the patient's vasculature, typically with the use of a dilator. FIG. 2 shows a deployment catheter 16 extending from the SVC 15 to the coronary sinus 19 of the heart 1, the deployment catheter 16 having been passed through the introducer sheath which provides a hemostatic valve to prevent blood loss.

In one embodiment, the deployment catheter 16 may be about 30 cm long, and the guidewire may be somewhat longer for ease of use. In some embodiments, the deployment catheter may function to form and prepare an opening in the wall of the left atrium 2, and a separate placement or delivery catheter will be used for delivery of an expandable shunt. In other embodiments, the deployment catheter may be used as the both the puncture preparation and shunt placement catheter with full functionality. In the present application, the terms “deployment catheter” or “delivery catheter” will be used to represent a catheter or introducer with one or both of these functions.

Since the coronary sinus 19 is largely contiguous around the left atrium 2, there are a variety of possible acceptable placements for the stent. The site selected for placement of the stent, may be made in an area where the tissue of the particular patient is less thick or less dense, as determined beforehand by non-invasive diagnostic means, such as a CT scan or radiographic technique, such as fluoroscopy or intravascular coronary echo (IVUS).

Some methods to reduce LAP involve utilizing a shunt between the left atrium 2 and the right atrium 5, through the interatrial septum therebetween. This is a convenient approach, as the two structures are adjacent and transseptal access is common practice. However, there may be a possibility of emboli travelling from the right side of the heart to the left, which presents a stroke risk. This event should only happen if the right atrium pressures go above left atrium pressures; primarily during discrete events like coughing, sneezing, Valsalva maneuver, or bowel movements. The anatomical position of the septum would naturally allow emboli to travel freely between the atria if a shunt was present and the pressure gradient flipped. This can be mitigated by a valve or filter element in the shunt, but there may still be risk that emboli will cross over.

Shunting to the coronary sinus 19 offers some distinct advantages, primarily that the coronary sinus 19 is much less likely to have emboli present for several reasons. First, the blood draining from the coronary vasculature into the right atrium 5 has just passed through capillaries, so it is essentially filtered blood. Second, the ostium of the coronary sinus 19 in the right atrium 5 is often partially covered by a pseudo-valve called the Thebesian Valve. The Thebesian Valve is not always present, but some studies show it is present in >60% of hearts and it would act as a natural “guard dog” to the coronary sinus to prevent emboli from entering in the event of a spike in right atrium pressure. Third, pressure gradient between the coronary sinus 19 and the right atrium 5 into which it drains is very low, meaning that emboli in the right atrium 5 is likely to remain there. Fourth, in the event that emboli do enter the coronary sinus 19, there will be a much greater gradient between the right atrium 5 and the coronary vasculature than between the right atrium 5 and the left atrium 2. Most likely emboli would travel further down the coronary vasculature until right atrium pressure returned to normal and then the emboli would return directly to the right atrium 5.

Some additional advantages to locating the shunt between the left atrium 2 and the coronary sinus 19 is that this anatomy is less mobile than the septum (it is more stable), it thus preserves the septum for later transseptal access for alternate therapies, and it could potentially have other therapeutic benefits. By diverting left atrial blood into the coronary sinus 19, sinus pressures may increase by a small amount. This would cause blood in the coronary vasculature to travel more slowly through the heart, increasing perfusion and oxygen transfer, which would be more efficient and also could help a dying heart muscle to recover. There is a device designed to do this very thing, the Neovasc Reducer. The preservation of transseptal access also is a very significant advantage because HF patients often have a number of other comorbidities like Atrial Fibrillation (AF) and Mitral Regurgitation (MR) and several of the therapies for treating these conditions require a transseptal approach.

A shunt may also be positioned between other cardiac chambers, such as between the pulmonary artery and right atrium 5. The shunt may be desirably implanted within the wall of the pulmonary artery using the deployment tools described herein, with the catheters approaching from above and passing through the pulmonary artery. As explained above, pulmonary hypertension (PH) is defined as a rise in mean pressure in the main pulmonary artery. Blood flows through the shunt from the pulmonary artery into the right atrium 5 if the pressure differential causes flow in that direction, which attenuates pressure and reduces damage to the pulmonary artery. The purpose is to attenuate pressure spikes in the pulmonary artery. The shunt may also extend from the pulmonary artery to other heart chambers (e.g., left atrium 2) and/or blood vessels. Although not preferred or shown, the shunt may further contain a one-way valve for preventing backflow, or a check valve for allowing blood to pass only above a designated pressure. The present application discloses a new shunt for maintaining a flow path between chambers of the heart. Some shunts described herein may be at least partially expandable. Moreover, in some embodiments, a shunt may have various features and/or may be used in combination with devices having various barriers for preventing, inhibiting, and/or containing tissue growth. As used herein, the term “barrier” is used according to its broad and ordinary meaning and may include any feature of a shunt and/or configured to be used in conjunction with a shunt to at least partially prevent, inhibit, reduce, contain, and/or otherwise alter tissue growth at and/or around the shunt. Shunts described herein may have various features to simplify and/or improve delivery procedures for surgeons. For example, a shunt may be at least partially flexible, compressible, and/or elastic to allow the shunt to be shaped and/or molded as necessary/desired to fit openings and/or tissue walls having various sizes and/or shapes.

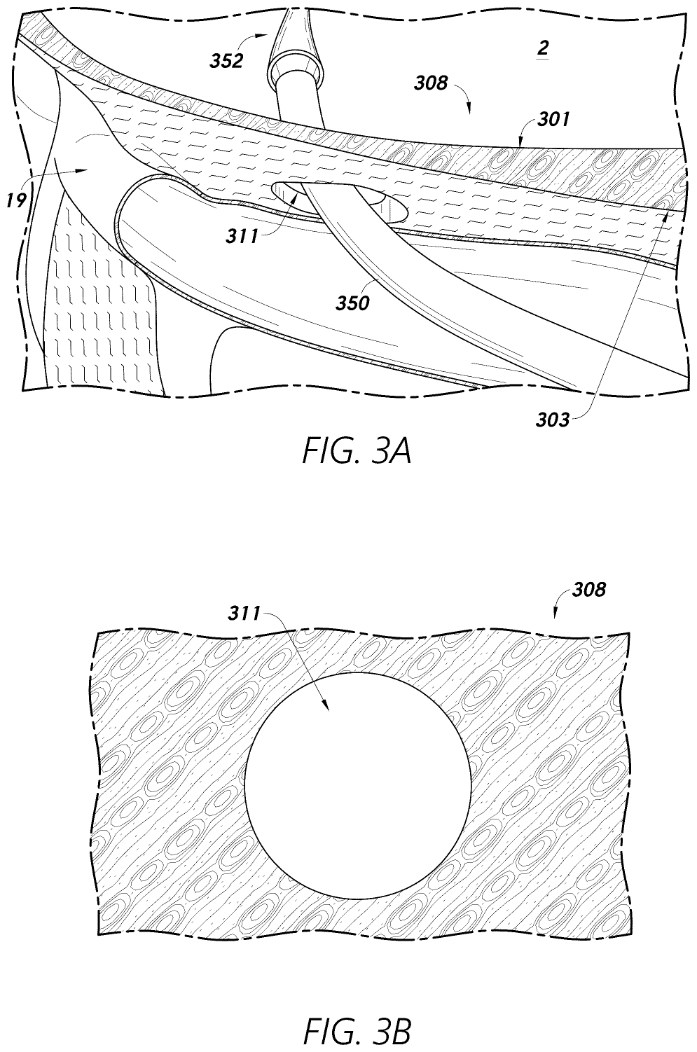

FIG. 3A is a side view and FIG. 3B is a view from above (e.g., from the left atrium 2) of an opening (i.e., puncture hole) 311 through a tissue wall 308 (e.g., between the coronary sinus 19 and the left atrium 2) for placement of a shunt in the opening 311. As shown in FIG. 3A, a shunt deployment or delivery catheter 350 may be advanced to the tissue wall 308 between two chambers (e.g., the coronary sinus 19 and the left atrium 2). A first side 301 of the tissue wall 308 may be situated on a side of a first anatomical chamber (e.g., the left atrium 2) and/or a second side 303 of the tissue wall 308 may be situated on a side of a second anatomical chamber (e.g., the coronary sinus 19). The catheter 350 may have a soft and/or tapered distal tip 352. The delivery catheter 350 may be advanced through the opening 311 in the tissue wall 308 into, for example, the left atrium 2. The opening may be created in any of a variety of ways. One example method is the following.

Initially, a guidewire may be advanced, for example, from the right atrium into the coronary sinus 19 through its ostium or opening. A puncture catheter may be advanced over the guidewire. The puncture catheter may be introduced into the body through a proximal end of an introducer sheath. An introducer sheath may provide access to the particular vascular pathway (e.g., jugular or subclavian vein) and may have a hemostatic valve therein. While holding the introducer sheath at a fixed location, the surgeon can manipulate the puncture catheter to the implant site. A puncture sheath having a puncture needle with a sharp tip may be advanced along a catheter and punctured through the wall 308 into, for example, the left atrium 2. A puncture expander may be advanced along the guidewire and through the tissue wall 308 into the left atrium 2. The puncture expander may be, for example, an elongated inflatable balloon. The puncture expander may be inflated radially outward so as to widen the puncture through the tissue wall 308.

A shunt may be delivered through a lumen of the catheter 350. During delivery, the shunt may be in a collapsed configuration to facilitate delivery. For example, the shunt may be rolled, bent, twisted, and/or otherwise configured to have a minimal profile to facilitate delivery through the catheter 350. The shunt may be located in the annular space between an inner sheath and outer sheath of the catheter 350. An inner sheath may be retracted so that the shunt is placed in intimate engagement with the tissue wall 308. Radiopaque markers may be provided to facilitate positioning of the catheter 350 and/or shunt. By creating an opening between the left atrium 2 and the coronary sinus 19, blood can flow from the left atrium 2 (which is usually >8 mmHg) to the coronary sinus 19 (which is usually <8 mmHg).

### Shunt Implants

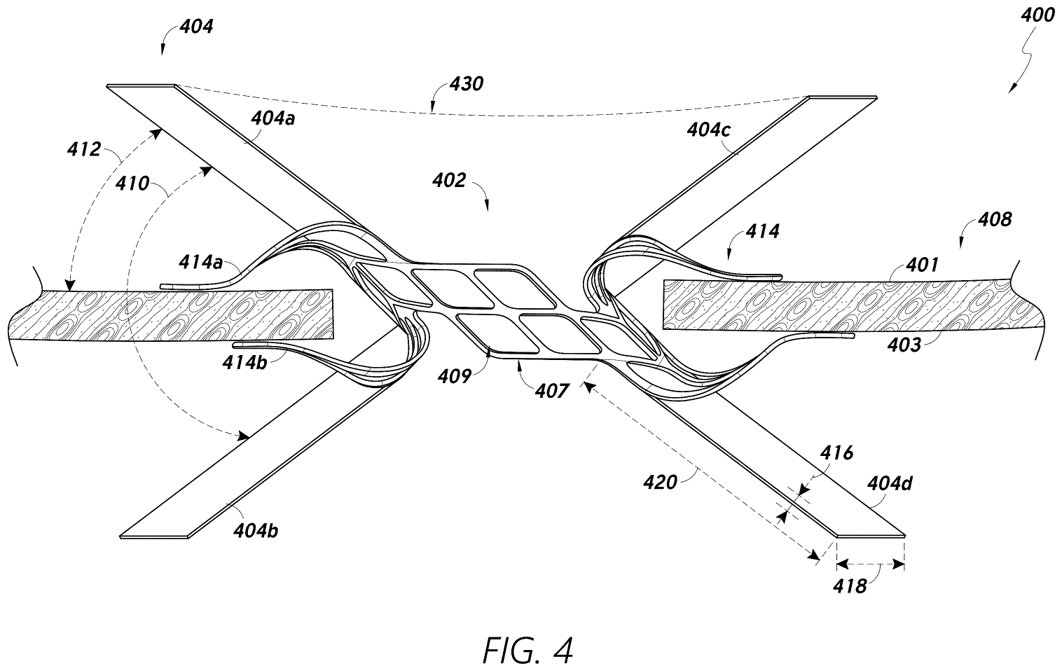

FIG. 4 illustrates a shunt 400 having one or more barrier walls 404 to prevent, contain, and/or inhibit tissue growth at and/or around the shunt 400 and/or an opening in a tissue wall in accordance with some embodiments. As used herein, the term “barrier wall” may refer to any portion of a material configured to form a barrier and/or obstruction between at least a portion of tissue and at least a portion of a shunt and/or opening through a tissue wall 408. The shunt 400 may comprise any of a variety of features and/or components configured to maintain an opening in a tissue wall 408 and/or allow blood flow through the tissue wall 408. In some embodiments, the shunt 400 may comprise a central flow portion 402 which may be configured to be situated at least partially within the opening in the tissue wall 408. In some embodiments, the shunt 400 may comprise multiple separate components which may be attached, connected, and/or otherwise joined to form a single device. For example, the central flow portion 402 may comprise multiple components to form a generally tubular shape which may approximate a shape of the opening in the tissue wall 408. For example, the opening may have a generally elliptical (e.g., circular) form (see, e.g., FIG. 3B) and the central flow portion 402 may be configured to form a generally cylindrical and/or tubular form to fit within and/or press against an inner surface of the tissue wall 408 at the opening.

The one or more walls 404 may be configured to extend outwardly from the central flow portion 402 and/or from one or more anchoring arms 414 of the shunt 400. For example, a wall 404 may extend from and/or attach to the central flow portion 402, however one or more walls 404 may extend from and/or attach to at least one of the one or more anchoring arms 414. In some embodiments, the one or more walls 404 may extend outwardly from the central flow portion 402 in a V-shape. For example, a first wall 404a may extend from the central flow portion 402 in a first direction (e.g., on a first side 401 of the tissue wall 408) and a second wall 404b may extend form the central flow portion 402 in a second direction (e.g., on a second side 403 of the tissue wall 408) to form a first angle 410 between the first wall 404a and the second wall 404b. For example, the first angle 410 may be approximately ninety degrees. In some embodiments, a wall 404 may comprise an elongate sheet that may be bent at a middle portion of the wall 404 to form a first portion (i.e., the first wall 404a) and a second portion (i.e., the second wall 404b) extending outwardly from the central flow portion 402 in different directions with a the first angle 410 of separation between the walls 404. Accordingly, the first wall 404a and the second wall 404b may comprise a single continuous device.

In some embodiments, a wall 404 may comprise a sheet of material having a relatively small thickness 416 in comparison to a width 418 of the wall 404 and/or may have a relatively small thickness 416 and/or width 418 in comparison to a length 420 of the wall 404. However, a wall 404 may have any thickness 416, width 418, and/or length 420. Each wall 404 may have a common thickness 416, width, and/or length 420 or individual walls 404 may have different thicknesses 416, widths, and/or lengths 420. As shown in FIG. 4, the shunt 400 may comprise multiple distinct walls 404 with separate and/or finite widths 418. However, in some embodiments, a wall 404 may have a generally conical shape in which the width 418 of the wall 404 extends in a generally elliptical form and forms a complete or near-complete ellipse. For example, the first wall 404a and/or a third wall 404c may each extend in a non-linear manner until they join to generally form a cone shape. For example, a wall 404 may have a generally elliptical form in which the wall 404 forms a complete or near complete ellipse of tissue over the tissue wall (e.g., a full ellipse of tissue at the first side 401 of the tissue wall 408). Accordingly, the first wall 404a and the third wall 404c may extend laterally (e.g., along a first line 430) to form a single continuous wall 404 (e.g., having an at least partial cone shape) around the first side 401 of the tissue wall 408. Similarly, the second wall 404b and the fourth wall 404d may extend to from a single continuous wall. Moreover, the wall 404 may have a generally tapered shape in which a diameter of the wall 404 increases as the wall 404 extends further from the central flow portion 402 and/or the opening. The wall 404 may only have a partial cone shape because the wall 404 may have a hollow middle portion configured to be aligned with the opening in the tissue wall 408. Accordingly, the wall 404 may not extend over the opening in the tissue wall 408.

Moreover, the first wall 404a, second wall 404b, third wall 404c, and a third wall 404d may form a double cone or a partial double cone in which the apex of the double cone may be a true apex at or near the central flow portion 402 and/or in which there is no apex point but rather the double cone form of the wall 404 may have a hollow middle portion which approximates a tubular form of the central flow portion 402 and/or the opening in the tissue wall 408.

A wall 404 may be configured to prevent, inhibit, and/or contain tissue growth around the shunt 400. Each wall 404 may extend outward form the central flow portion 402 over a portion of the tissue wall 408. For example, the first wall 404a may extend over a portion of the first side 401 of the tissue wall 408. As shown in FIG. 4, the first wall 404a may extend at a second angle 412 from the first side 401 of the tissue wall 408. In some embodiments, the first wall 404a may extend in a generally parallel or perpendicular direction with respect to the tissue wall 408. While the first wall 404a and/or other walls 404 are shown having a generally linear form, each wall 404 may have a curvature and/or may be bent at various points as desired. A wall 404 may be configured to extend over the tissue wall 408 (e.g., the first side 401 of the tissue wall 408) at any angle. For example, the wall 404 may be configured to extend such that the second angle 412 between the first wall 404a and the first side 401 of the tissue wall is approximately 45-degrees.

In some embodiments, a wall 404 may be configured to at least partially penetrate and/or pass through the tissue wall 408. For example, a wall 404 may extend outwardly from the central flow portion 402 and into the tissue wall 408. The wall 404 may be partially embedded in the tissue wall 408 and/or a portion of the tissue wall 408 may extend out of the tissue wall 408.

As the tissue wall 408 increases in thickness and/or otherwise expands (e.g., grows inwardly towards the central flow portion 402), the tissue may press against the walls 404. The walls 404 may be composed of an at least partially solid material and/or a sufficiently densely interconnected network of materials that tissue growth through the walls 404 may be prevented and/or at least partially inhibited.

Any of the one or more anchoring arms 414 may comprise one or more anchoring mechanisms, which may be situated, for example, at an end portion of the anchoring arm. Suitable anchoring mechanism may include any devices configured to penetrate and/or otherwise securely contact the tissue wall. For example, an anchoring mechanism may comprise one or more of a barb, a hook, a nail, and a screw. When the shunt 400 is placed at the tissue wall 408, the anchoring mechanisms may be configured to interact with the tissue wall 408 to securely hold the shunt 400 in place.

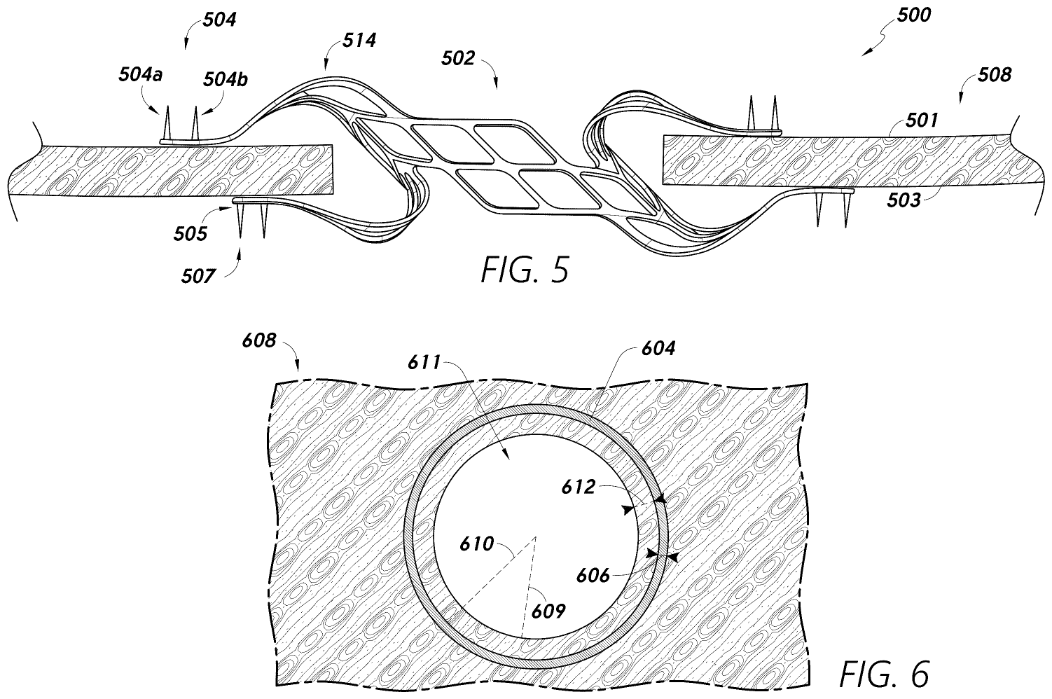

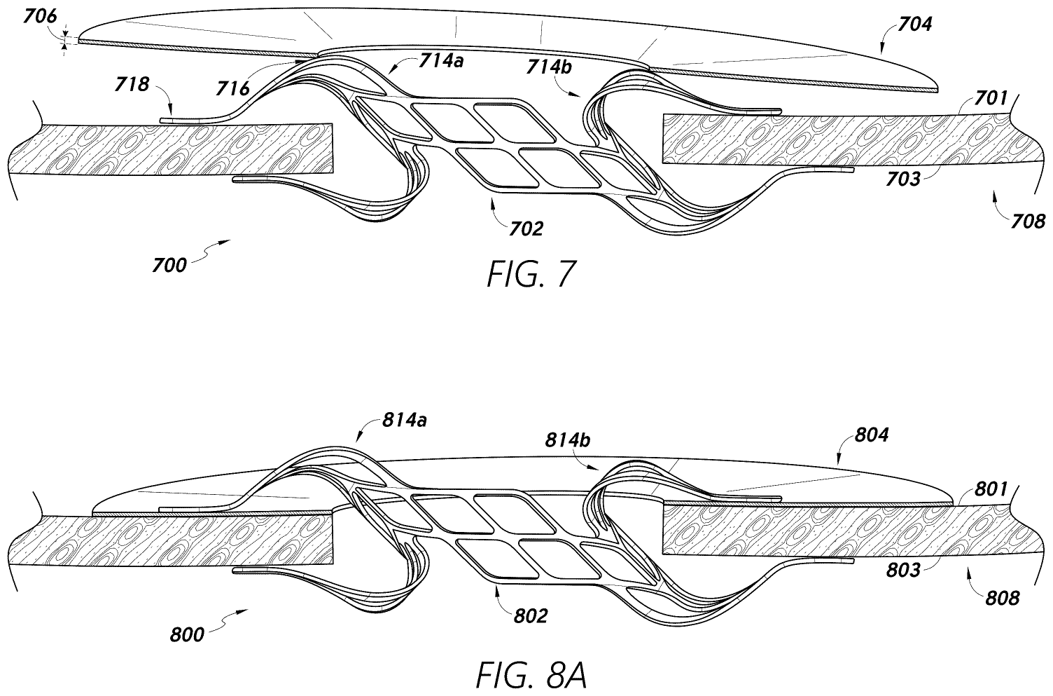

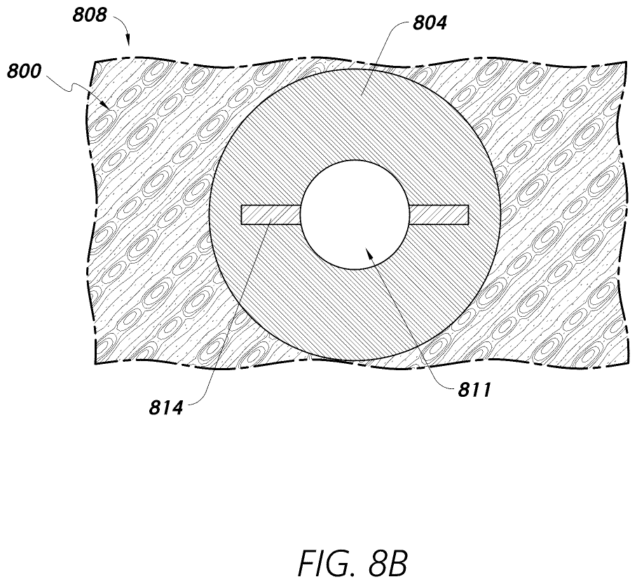

Various features of the shunt 400, including the central flow portion 402 and/or anchoring arms 414 described herein may be applied to the shunt devices described and/or illustrated in other figures of the present application. For example, any description with respect to the shunt 400 illustrated in FIG. 4 may be similarly applied to the shunt 500 in FIG. 5, the shunt 700 in FIG. 7, and the shunt 800 in FIGS. 8A and 8B described herein. Moreover, while other shunts shown and/or described with respect to other figures may not include walls 404 as shown in FIG. 4, it will be understood that walls 404 may be added to the shunts described with respect to other figures. Similarly, the various features described with respect to other figures herein may be added to the shunt 400 of FIG. 4 and/or other figures herein even if not depicted in and/or described with respect to each figure.

In some embodiments, the shunt 400 may be configured to be movable between an expanded configuration and a collapsed (e.g., generally tubular) configuration to facilitate passage through a lumen of a catheter. For example, the central flow portion 402 may be configured to be rolled, bent, twisted, or otherwise compacted to fit within the lumen of the catheter. The central flow portion 402 may be configured to expand to a pre-defined shape and/or size during and/or after delivery within the body. The shunt 400 may further comprise one or more anchoring arms 414, which may include flanges, arms, anchors, and/or other devices. The one or more anchoring arms 414 may be configured to at least partially collapse to facilitate passage through the lumen of the catheter and may be configured to expand during and/or after delivery within the body to contact and/or attach to the tissue wall 408. Expansion of the shunt 400 may be initiated, for example, by retraction of an outer sheath of the catheter relative to an inner sheath. The shunt 400 may be collapsed (e.g., crimped) into a generally tubular configuration between the two sheaths with the anchoring arms 414 straightened, and the anchoring arms 414 may be configured to spring open when the restraining outer sheath retracts. The anchoring arms 414 may expand generally in opposite direction in a common plane to form a T-shape, as opposed to expanding in a circular fashion. Radiopaque markers on the anchoring arms 414 may be provided to facilitate positioning immediately within the left atrium.

A pair of anchoring arms 414 (e.g., a first anchoring arm 414a and a second anchoring arm 414b) may form a clamping (i.e., pinching) pair of anchoring arms. The pairs of anchoring arms 414 may be configured to apply a compressive force to the tissue wall 408 to hold the shunt 400 in place. The amount of compressive force may be relatively small to avoid damage to the tissue wall 408 while sufficient to hold the shunt 400 in place. For example, gaps separating the pairs of anchoring arms may be calibrated to avoid excessive clamping and/or necrosis of the tissue. The anchoring arms 414 may be configured to secure the shunt 400 on generally opposite sides of the tissue wall 408 (e.g., the first anchoring arm 414a on a first side 401 of the tissue wall 408 and the second anchoring arm 414b on a second side 403 of the tissue wall 408) and/or on generally opposite sides of the opening in the tissue wall 408. The central flow portion 402 may be configured to be aligned generally perpendicular to the tissue wall 408 so as to maintain an open flow path between the chambers on either side of the tissue wall 408 (e.g., the coronary sinus and the left atrium).

Components of the shunt 400 may be configured to naturally self-expand due to inherent springiness and/or flexibility of the components. For example, various components (e.g., the central flow portion 402, anchoring arms 414, and/or walls 404) may be composed of an elastic material such as Nitinol. In some embodiments, the central flow portion 402 may be fabricated by laser cutting a Nitinol tube. The central flow portion 402 may have a wall thickness of between about 0.1-0.3 mm.

As shown in FIG. 4, the central flow portion 402 may be composed of generally thin struts 407 in a generally parallelogram arrangement that may form an array of parallelogram-shaped cells 409 or openings. However, the central flow portion 402, including the struts 407 and/or cells 409, may have any shape, size, and/or orientation. For example, the struts 407 may have a generally thicker design than shown in FIG. 4 to minimize the size of the cells 409, thereby further preventing in-growth of tissue through the central flow portion 402. Rather than a generally parallelogram shape, the cells 409 may have a generally elliptical, triangular, hexagonal, or other shape. Moreover, the central flow portion 402 may not comprise any cells 409. In some embodiments, the shape of the struts 407, cells 409, and/or the central flow portion 402 generally may facilitate a collapsibility and/or expandability of the central flow portion 402 for passage through a lumen of a catheter.

The flow portion 402 may be configured to form a generally cylindrical or other shape to approximate a shape of the opening. In some embodiments, the opening may be widened in all directions approximately evenly from a puncture point to form an approximately circular opening having a certain diameter. Accordingly, the flow portion 402, including the struts 407, may have an at least partially rounded and/or circular form around/about the opening along a longitudinal axis (i.e., into the opening).

In some embodiments, the expandable shunt 400 may be in a compacted and/or otherwise expandable form at delivery. For example, at delivery, the central flow portion 402, anchoring arms 414, and/or walls 404 may be folded, bent, and/or otherwise compacted to have a minimal profile to facilitate passage through a delivery catheter. After delivery, the central flow portion 402, anchoring arms 414, and/or walls 404 may be configured to unfold, unwrap, and/or otherwise expand (e.g., to form the design shown in FIG. 4). In some embodiments, at least a portion of the central flow portion 402, anchoring arms 414, and/or walls 404 may be composed of Nitinol and/or a similar material having shape-memory characteristics such that the shunt 400 may naturally assume a pre-determined form after removal from the delivery catheter.

Moreover, the central flow portion 402 and/or anchoring arms 414 may be configured to expand in response to growth and/or expansion of the tissue wall 408. For example, as the tissue wall 408 expands (i.e., thickens), the first anchoring arm 414a and the second anchoring arm 414b may be configured to separate further from each other to some extent to accommodate the growth of the tissue wall 408. In some embodiments, the central flow portion 402, anchoring arms 414, and/or walls 404 may be configured to stretch in response to expansion of the tissue wall 408. For example, the central flow portion 402, anchoring arms 414, and/or walls 404 may be at least partially composed of a flexible and/or elastic material that may allow for some amount of stretching. As the tissue wall 408 expands, the shunt 400 may be configured to stretch to accommodate the expansion of the tissue wall 408.

While each of FIGS. 4-8 may illustrate medical implants and/or processes including features for preventing, containing, and/or inhibiting tissue growth at or near medical implants, these features may be used independently of each other or in combination with each other. For example, a shunt 400 as shown in FIG. 4 may include walls 404 for managing tissue growth without any additional features for managing tissue growth. Alternatively, for example, the walls 404 and/or other features describes herein may be utilized in combination with other features. For example, the shunt 400 may comprise one or more spikes (as shown in FIG. 5) and/or barriers (as shown in FIGS. 7 and 8) extending from the walls 404 and/or other areas of the shunt 400. As another example, the tissue wall 408 may be treated (as shown in FIG. 6) prior to delivery of the shunt 400 and/or any other shunt described herein.

FIG. 5 illustrates a shunt 500 having one or more barrier spikes 504 to prevent, contain, and/or inhibit tissue growth in accordance with some embodiments. The shunt 500 may comprise a central flow portion 502 and/or one or more anchoring arms 514, similar to the central flow portion 502 and anchoring arms 514 described above with respect to FIG. 4. Anchoring arms 514 may be configured to extend from the central flow portion 502 to contact and/or attach to a first side 501 and/or second side 503 of the tissue wall 508.

The shunt 500 may further comprise one or more spikes 504, which may include needles, rods, bumps, and/or other protuberances which may extend from anchoring arms 514 and/or the central flow portion 502. While FIG. 5 shows two spikes 504 extending from each anchoring arm 514, any number of spikes 504 may extend from an anchoring arm 514 and/or one or more spikes 504 may extend from the central flow portion 502. In some embodiments, a spike 504 may be composed of a different material than the anchoring arms 514 and/or central flow portion 502 and/or a spike 504 may represent a separate component from the anchoring arms 514 and/or central flow portion 502 and may be attached to the anchoring arms 514 and/or central flow portion 502.

In some embodiments, a spike 504 may be a generally thin device which may have a base portion 505 that is in contact with an anchoring arm 514 and/or central flow portion 502. From the base portion 505, the spike 504 may extend to an end portion 507. In some embodiments, a distance from the base portion 505 to the end portion 507 (i.e., a length of the spike 504) may exceed a thickness of the spike 504. However, a spike 504 may have any thickness and a length of the spike 504 may be less than a thickness of the spike 504. While the spikes 504 are shown extending generally perpendicularly to the tissue wall 508, the spikes 504 may extend from the shunt 500 and/or from the tissue wall 508 at any angle. For example, a spike 504 may extend in a diagonal direction away from or towards the central flow portion 502 and/or opening.

The end portion 507 of a spike 504 may be generally pointed, rounded, and/or may have any other shape. In some embodiments, the end portion 507 may be sufficiently pointed that the end portion 507 may be capable of piercing tissue. For example, as a tissue wall 508 grows/expands, tissue may extend at least partially over an anchoring arm 514 and/or the central flow portion 502. As the tissue encounters a spike 504, the spike 504 may be sufficiently rigid that the tissue is not able to push through the spike 504 and may be required to grow over the spike 504. As the tissue extends over the end portion 507 of the spike 504, the end portion 507 may be configured to pierce and/or press against the tissue to cause the tissue to recede and/or stop growing over the shunt 500 in at least one direction.

In some embodiments, spikes 504 may be positioned in multiple levels. For example, a first spike 504a may be positioned near a distal end of an anchoring arm 514 (i.e., distal from the central flow portion 502) and a second spike 504b may be positioned near the central flow portion 502. That is, the first spike 504a may be further from the central flow portion 502 and/or the opening than the second spike 504b. As the tissue wall 508 grows/expands, tissue may encounter the second spike 504b after passing over the first spike 504a.

A spike 504 may have various features for piercing and/or otherwise inhibiting tissue growth. For example, a spike 504 may include multiple smaller spikes which may extend generally perpendicularly from the spike 504. Accordingly, as tissue grows over the spike 504, the smaller spikes may pierce and/or press against the tissue to represent an additional barrier to the tissue. Similarly, a spike 504 may have a ridged surface and/or may be coated in a sand-like or similar abrasive material to present an obstacle to tissue growth.

Various embodiments and/or features of embodiments described herein may be combined. For example, one or more spikes 504 may extend from a wall 404 described herein with respect to FIG. 4.

FIG. 6 illustrates a method of preventing, inhibiting, reducing, and/or containing tissue growth involving treating one or more areas 604 of tissue around and/or near an opening 611 through a tissue wall 608 in accordance with some embodiments. The method may involve burning, cutting, removing, cauterizing, scarring, and/or otherwise treating the one or more areas 604 of tissue. An area 604 of tissue may comprise a portion of an outer surface of a tissue wall 608 (e.g., on a left atrium side or coronary sinus side of the tissue wall 608) and/or on an inner surface of the tissue wall 608 (e.g., within the opening 611 in the tissue wall 608).

In some embodiments, one or more areas 604 of tissue may be treated prior to, during, and/or after placement of a shunt at or near the opening 611. Various tools may be delivered for use in treating one or more areas 604 of tissue. For example, a laser or similar device may be used to remove and/or burn the area 604 of tissue. Treatment of the one or more areas 604 may involve electrical ablation and/or use of an electrical cauterizing tool to cause a controlled scarring pattern and/or block electrical transmission at an area 604 of tissue.

As shown in FIG. 6, the area 604 may have an elliptical (e.g., circular) shape and/or may approximate a shape of the opening 611 in the tissue wall 608. For example, the opening 611 may have a generally circular shape having a first radius 609 and the area 604 may similarly have a generally circular shape having a second radius 610 which may be greater than the first radius 609. However, the one or more areas 604 may have any size and/or shape. For example, an area 604 may not comprise a complete ellipse and may instead comprise a linear, jagged, curved, non-linear, etc. shape that may be situated at or near an anchoring location of a shunt implant. In some embodiments, multiple areas 604 may be created. For example, multiple elliptical or semi-elliptical areas 604 having different sizes and/or radii may form an elliptical or other shape. The one or more areas 604 may represent multiple levels of treated tissue along a lateral axis extending from the opening 611. For example, a first area 604 of tissue may have a wavy shape in which the first area 604 overlaps itself one or more times along a single axis extending from the opening 611. In another example, a first area 604 may be positioned a first distance along a lateral axis from the opening 611 and a second area 604 may be positioned a second distance along the lateral axis from the opening 611, in which the second distance is greater than the first distance. In other words, the second area 604 may be positioned distal to the opening 611 and the first area 604 may be positioned proximal to the opening 611. In some embodiments, an area 604 may have any thickness 606 and/or may have a gap 612 of any size between the opening 611 and the area 604.

In some embodiments, an area 604 may be treated in conjunction with delivery of a shunt as described herein. A shunt may be placed at least partially within the opening 611. The shunt may have one or more anchoring arms configured to extend over and/or contact the tissue wall 608 around the opening 611. In some embodiments, an anchoring arm of a shunt may be configured to extend over an area 604 or not extend beyond the gap 612 between the opening 611 and the area 604. The shape and/or size of an area 604 may be selected based on a shape and/or size of a shunt placed at the opening 611. For example, before and/or after a shunt is placed, an area 604 shaped to closely surround at least a portion of the shunt may be treated. In this way, the area 604 may be configured to prevent growth and/or in-growth of tissue at and/or around the shunt.

FIG. 7 illustrates a shunt 700 having an upper barrier 704 to prevent, contain, and/or inhibit tissue growth in accordance with some embodiments. In some embodiments, the upper barrier 704 may have an elliptical and/or torus shape. The upper barrier 704 may form a complete or partial ring around a hollow middle portion of the ring. The hollow middle portion may be configured to be aligned with the opening in the tissue wall 708 and/or a flow path created and/or maintained by a central flow portion 702 of the shunt 700. For example, the central flow portion 702 may define a flow path through a tissue wall 708 and the upper barrier 704 may be configured to surround but not obstruct (or only partially obstruct) the flow path.

Because FIG. 7 shows a cross-sectional view of the shunt 700, the upper barrier 704 is shown as having a partial elliptical shape. However, the upper barrier 704 may form a complete ellipse around the opening in the tissue wall 808. The upper barrier 704 may have any shape. For example, the upper barrier 704 may have a rectangular, triangular, pentagonal, octagonal, or other shape and/or may include a hole through a middle portion of the upper barrier 704 to allow flow through the upper barrier 704.

While the upper barrier 704 is shown having a generally thin structure, the upper barrier 704 may have any thickness 706. Moreover, the upper barrier 704 may have a varying thickness 706. For example, the upper barrier 704 may have an at least partially rounded surface in which a cross section of the upper barrier 704 would have an ellipse shape, similar to a torus.

In some embodiments, the upper barrier 704 may be configured to extend from and/or attach to the central flow portion 702 and/or to one or more anchoring arms 714 of the shunt 700. For example, the upper barrier 704 may attach to and/or extend from a first anchoring arm 714a and/or a second anchoring arm 714b. The first anchoring arm 714a and the second anchoring arm 714b may be situated on generally opposite sides of the opening on a first side 701 of the tissue wall 708 (or a second side 703 of the tissue wall 708). In some embodiments, the upper barrier 704 may represent a portion of the shunt 700 that is furthest from the first side 701 of the tissue wall 708 and/or the second side 703 of the tissue wall 708.

While only a single upper barrier 704 is shown in FIG. 7, the shunt 700 may comprise multiple upper barriers 704. For example, the shunt 700 may comprise a second upper barrier 704 extending from and/or attaching to the central flow portion 702 and/or one or more anchoring arms 714 at the second side 703 of the tissue wall 708. Moreover, while the upper barrier 704 is shown extending from and/or attaching to one or more proximal portions 716 of the anchoring arms 714, the upper barrier 704 may be configured to extend from and/or attach to any portion(s) of the anchoring arms 714 and/or central flow portion 702. For example, the upper barrier 704 may be configured to attach to and/or extend from one or more end portion 718 of the anchoring arms 714. In some embodiments, the hole in the middle portion of the upper barrier 704 may be sufficiently large that one or more proximal portions 716 of the anchoring arms 714 may be configured to fit into and/or through the hole when the upper barrier 704 is configured to extend from and/or attach to the end portions 718 of the anchoring arms 714.

While the upper barrier 704 is shown having a generally flat structure, the upper barrier 704 may have any shape and/or size. For example, the upper barrier 704 may have a generally wavy structure in which a high point (i.e., peak) of the upper barrier 704 is configured to fit over a proximal portion 716 of an anchoring arm and a low point (i.e., trough) of the upper barrier 704 is configured to be situated close to and/or to press against the tissue wall 708. In some embodiments, the upper barrier 704 may be composed of a generally flexible and/or elastic material such that the upper barrier 704 may be configured to at least partially bend around portions of the anchoring arms 714 and/or central flow portion 702 to fit closely to the anchoring arms 714 and/or central flow portion 702 and/or to minimize gaps around and/or through the anchoring arms 714 and/or central flow portion 702. For example, the upper barrier 704 may have a generally soft structure and/or may be configured to approximate contours of the anchoring arms 714 and/or central flow portion 702 and/or to approximate a general shape of the anchoring arms 714 and/or central flow portion 702 when placed and/or situated on top of the anchoring arms 714 and/or central flow portion 702. In some embodiments, the upper barrier 704 may be sufficiently rigid in structure that it may at least partially resist growth and/or expansion of the tissue wall 708.

FIGS. 8A and 8B illustrate a shunt 800 having a lower barrier 804 to prevent, contain, reduce, and/or inhibit tissue growth in accordance with some embodiments. FIG. 8A provides a side-view of the shunt 800 and FIG. 8B provides a view of the shunt 800 from above (e.g., from the left atrium). In some embodiments, the lower barrier 804 may have an elliptical and/or torus shape. The lower barrier 804 may form a complete ring around a central hole, which may align with a flow path created and/or maintained by a central flow portion 802 of the shunt 800. For example, the central flow portion 802 may define a flow path through a tissue wall 808 and the upper barrier 804 may be configured to surround but not obstruct (or only partially obstruct) the flow path.

Because FIG. 8A shows a cross-sectional view of the shunt 800, the lower barrier 804 is shown as having a partial elliptical shape. However, the lower barrier 804 may form a complete ellipse around the opening in the tissue wall 808. The lower barrier 804 may have any shape. For example, the lower barrier 804 may have a rectangular, triangular, pentagonal, octagonal, or other shape and/or may include a hole through a middle portion of the lower barrier 804 to allow flow through the lower barrier 804.

The lower barrier 804 may be configured to be situated between one or more anchoring arms 814 and the tissue wall 808. For example, one or more anchoring arms 814 may be configured to press the lower barrier 804 against the tissue wall 808. While the lower barrier 804 is shown having a generally thin structure, the lower barrier 804 may have any thickness. Moreover, the lower barrier 804 may have a varying thickness. For example, the lower barrier 804 may have an at least partially rounded surface in which a cross section of the lower barrier 804 would have an ellipse shape, similar to a torus.

In some embodiments, the lower barrier 804 may be configured to extend from and/or attach to the central flow portion 802 and/or to one or more anchoring arms 814 of the shunt 800. For example, the lower barrier 804 may attach to and/or extend from a first anchoring arm 814a and/or a second anchoring arm 814b. The first anchoring arm 814a and the second anchoring arm 814b may be situated on generally opposite sides of the opening 811 on a first side 801 of the tissue wall 808 (or a second side 803 of the tissue wall 808). In some embodiments, the central flow portion 802 and/or anchoring arms 814 may be configured to hold the lower barrier 804 in place by pressing the lower barrier 804 against the tissue wall 808. Additionally or alternatively, the lower barrier 804 may have various features configured to interact with the tissue wall 808 to hold the lower barrier 804 in place. For example, the outer surface of the lower barrier 804 may have a ridged and/or contoured structure configured to grip and/or penetrate the surface of the tissue wall 808. In another example, the lower barrier 804 may comprise one or more nails, screws, hooks, barbs, and/or other features configured to attach to and/or penetrate the tissue wall to securely hold the lower barrier 804 in place. In some embodiments, anchoring elements (e.g., nails, screws, hooks, barbs) may be separately delivered for anchoring the lower barrier 804 to the tissue wall 808.

While only a single lower barrier 804 is shown in FIGS. 8A and 8B, the shunt 800 may comprise multiple lower barriers 804. For example, the shunt 800 may comprise a second lower barrier 804 pressed against the second side 803 of the tissue wall 808. The lower barrier(s) 804 may have a sufficiently rigid structure to oppose and/or resist growth of tissue at and/or around the lower barrier 804.

### Delivery Processes

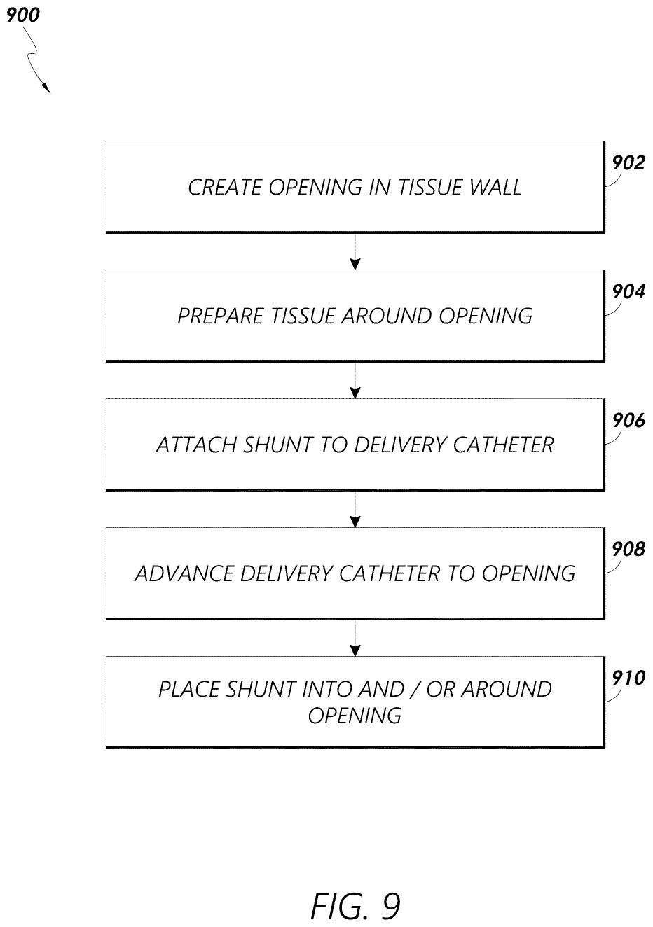

FIG. 9 is a flow diagram of an example of a process 900 for delivering and/or anchoring a shunt to a body of a person to in accordance with some embodiments. In block 902, the process 900 involves creating an opening in a tissue wall. As described herein, the opening may be created through use of one or more of a guidewire, puncture catheter, introducer sheath, puncture sheath, and/or puncture expander. The opening may create a blood flow path between two anatomical chambers (e.g., the left atrium and the coronary sinus). The opening may be created in any of a variety of ways. One example method is the following.

Initially, a guidewire may be advanced, for example, from the right atrium into the coronary sinus through its ostium or opening. A catheter may be advanced over the guidewire. The catheter may be introduced into the body through a proximal end of an introducer sheath. An introducer sheath may provide access to the particular vascular pathway (e.g., jugular or subclavian vein) and may have a hemostatic valve therein. While holding the introducer sheath at a fixed location, the surgeon can manipulate the puncture catheter to the implant site. A puncture sheath having a puncture needle with a sharp tip may be advanced along a catheter and punctured through the wall into, for example, the left atrium. A puncture expander may be advanced along the guidewire and through the tissue wall into the left atrium. The puncture expander may be, for example, an elongated inflatable balloon. The puncture expander may be inflated radially outward so as to widen the puncture through the tissue wall. In some embodiments, the opening may have a generally circular shape.

An implant may be delivered through a lumen of the catheter. During delivery, the implant may be in a collapsed configuration to facilitate delivery. For example, the implant may be bent, twisted, and/or otherwise configured to have a minimal profile to facilitate delivery through the catheter. The implant may be located in the annular space between an inner sheath and outer sheath of the catheter. An inner sheath may be retracted so that the implant is placed in intimate engagement with the tissue wall. Radiopaque markers may be provided to facilitate positioning of the catheter and/or implant. By creating an opening between the left atrium and the coronary sinus, blood can flow from the left atrium (which is usually >8 mmHg) to the coronary sinus (which is usually <8 mmHg). One or more implants may be delivered and/or anchored to a first side and/or to a second side of the tissue wall 808.

In block 904, the process 900 involves preparing an area of tissue around the opening. In some embodiments, preparing the tissue may involve burning, scarring, and/or otherwise treating the tissue to prevent, inhibit, and/or contain tissue growth at and/or around the area of tissue. In some embodiments, the treated area may completely surround the opening in the tissue wall. For example, the treated area may form a circular (or other shape) area around the opening such tissue growth around the entire opening may be managed.

In block 906, the process 900 involves attaching a shunt to a delivery catheter. The shunt may be crimped onto an outer surface of the catheter and/or within a lumen of the delivery catheter and/or may be in a collapsed state during delivery. In some embodiments, the shunt may be configured to be situated between an outer surface of the catheter and a delivery sheath configured to at least partially cover the shunt. The sheath may be configured to at least partially prevent expansion of the shunt during delivery through various pathways of the body.

In block 908, the process 900 involves advancing the delivery catheter to and/or near the opening. In some embodiments, the shunt may be configured to at least partially bend to facilitate delivery to and/or near the opening. For example, the catheter and/or shunt may be at least partially bent to maneuver the catheter into the coronary sinus ostium and/or into the opening.

In block 910, the process 900 involves placing the shunt into and/or around the opening. For example, the shunt may comprise a flow portion and/or tube configured to be situated within the opening and/or one or more anchoring mechanisms configured to anchor the flow portion to portions of the tissue wall outside the opening. In some embodiments, the shunt may comprise various barriers configured to prevent, inhibit, reduce, and/or contain growth of tissue around the opening and/or around the shunt.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A method of forming an opening between a left atrium and a coronary sinus for reducing left atrial pressure, the method comprising:
advancing a guidewire through the right atrium and into the coronary sinus;
delivering a puncture catheter and an electrical cauterizing tool through an introducer sheath and over the guidewire, wherein the puncture catheter includes a sharp tip and at least one radiopaque marker;
positioning a distal end portion of the puncture catheter at a target location within the coronary sinus;
advancing the puncture catheter to create the opening in a wall between the coronary sinus and the left atrium;
treating an area of tissue around the opening using the electrical cauterizing tool to cause a controlled scarring pattern at the area of tissue; and
removing the puncture catheter and the electrical cauterizing tool from the coronary sinus;
wherein the opening creates a blood flow path between the left atrium and the coronary sinus for reducing left atrial pressure, thereby alleviating symptoms of diastolic heart failure.

2. The method of claim 1, further comprising placing a shunt within the opening.

3. The method of claim 1, wherein the guidewire is advanced through a femoral vein, an inferior vena cava, and into the right atrium.

4. The method of claim 1, wherein the guidewire is advanced through a superior vena cava and into the right atrium.

5. The method of claim 1, further comprising expanding the opening using a balloon.

6. The method of claim 5, wherein the balloon is in a deflated form during delivery and is inflated to an inflated form while positioned within the opening.

7. The method of claim 1, further comprising dilating the opening before treating the area of tissue.

8. A method of forming an opening between a left atrium and a coronary sinus of a heart, the method comprising:
delivering a puncture catheter, a puncture expander, and an ablation device to a target location within a coronary sinus of a heart via a catheter;
puncturing a wall between the coronary sinus and a left atrium to form an opening in the wall using the puncture catheter;
expanding the opening using the puncture expander;
treating an area of tissue within the opening using the ablation device to maintain the expanded opening; and
removing the puncture catheter, puncture expander, and ablation device from the heart.

9. The method of claim 8, further comprising delivering the puncture catheter and the ablation device to the target location within the coronary sinus via a right atrium of the heart.

10. The method of claim 8, further comprising advancing a guidewire the target location within the coronary sinus, wherein delivering the puncture catheter involves advancing the puncture catheter over the guidewire.

11. The method of claim 8, wherein the ablation device comprises an electrical cauterizing tool and wherein treating the area of tissue within the opening involves scarring the area of tissue within the opening using the electrical cauterizing tool.

12. The method of claim 8, wherein the puncture expander comprises an inflatable balloon.

13. The method of claim 8, wherein the opening is expanded before treating the area of tissue.

14. A method of forming an opening between a coronary sinus and a left atrium of a heart, the method comprising:
delivering a puncture catheter, a puncture expander, and an ablation device to a tissue wall between the coronary sinus and the left atrium via a catheter;
puncturing an opening at the tissue wall using the puncture catheter;
expanding the opening using the puncture expander;
treating one or more areas of tissue around the opening using the ablation device to prevent in-growth of tissue at the opening; and
removing the puncture catheter, puncture expander, and ablation device from the heart.

15. The method of claim 14, further comprising delivering the puncture catheter and the ablation device to the tissue wall within the coronary sinus via a right atrium of the heart.

16. The method of claim 14, further comprising advancing a guidewire to the tissue wall, wherein delivering the puncture catheter involves advancing the puncture catheter over the guidewire.

17. The method of claim 14, wherein the one or more areas of tissue are along a coronary sinus side of the tissue wall.

18. The method of claim 14, wherein the one or more areas of tissue are along a left atrium side of the tissue wall.

19. The method of claim 14, wherein there is a gap between the opening and a first area of tissue of the one or more areas of tissue.

20. The method of claim 14, wherein treating one or more areas of tissue involves treating a first area of tissue and treating a second area of tissue, wherein the first area of tissue is closer to the opening than the second area of tissue.
